(19) United States
(12) Patent Application Publication
(10) Pub. No.: US 2013/0116481 A1
(43) Pub. Date: May 9, 2013

(54) CONVERSION OF ALCOHOLS

(71) Applicants: Duncan Frank Wass, Westbury on Trym (GB); George Richard Michael Dowson, Bricklehampton (GB)

(72) Inventors: Duncan Frank Wass, Westbury on Trym (GB); George Richard Michael Dowson, Bricklehampton (GB)

(73) Assignee: BP Biofuels UK Limited, Middlesex (GB)

(21) Appl. No.: 13/809,111

(22) PCT Filed: Jul. 8, 2011
(86) PCT No.: PCT/GB2011/001036

§ 371 Date: Jan. 8, 2013

(30) Foreign Application Priority Data
Jul. 9, 2010 (EP) 10251236.5

(51) Int. Cl. C07C 29/32; B01J 31/24

(52) U.S. Cl. CPC C07C 29/32; B01J 31/2404
USPC 568/902; 502/155

(57) ABSTRACT

A method is described for use in a process for the conversion of an alcohol, the method including the step of contacting a composition comprising a first alcohol with a catalyst composition. Catalyst composition described comprises: i) a source of a Group VIII transition metal; ii) a phosphine ligand of formula PR¹R²R³, wherein R¹, R² and R³ are the same or different; and iii) a base. In examples described, the alcohol which is converted comprises ethanol and the product comprises butanol.

This invention relates to the conversion of alcohols. Aspects of the invention relate to the conversion of ethanol. Examples of aspects of the invention relate to the catalysed conversion of alcohols, for example ethanol. Examples of the invention described herein relate to the catalysed conversion of a first alcohol to a second alcohol, for example ethanol to butanol. Aspects of the invention relate to the production of alcohols. Aspects of the invention relate to a catalyst composition. Example catalyst compositions of the invention can be used in the production of an alcohol. Example catalyst compositions of the invention can be used in the conversion of an alcohol to a higher alcohol.

The desirability for alternative liquid fuels to fossil fuel-based materials has led to the emergence and development of so-called biofuels as a valuable alternative. Biofuels include fuels which are derived partly or fully from biomass and such fuels may be used as a replacement to a fossil fuel or may be present as a component blended with fossil fuels for example in gasoline. Biofuel alternatives or components for gasoline include ethanol and butanol. In general, the latter alcohol is considered to be a more advanced fuel because its fuel performance parameters, such as energy density, are greater than those of ethanol and are much closer to those of conventional gasoline. Butanol may also be blended into conventional gasoline in higher amounts compared with ethanol. Currently known processes for the manufacture of butanol largely rely on biosynthetic pathways from fermentable sugars. These routes currently have the disadvantage of low conversion rate. An alternative route is to convert biomass-derived ethanol into butanol by a catalysed reaction sequence, often called the ‘Guerbet’ or ‘borrowed hydrogen’ reaction (Dalton Transaction, 2009, pages 753-762). This reaction is described in the academic literature as having three stages: dehydrogenation of an alcohol (for example, ethanol) to an aldehyde (for example acetaldehyde); dimerisation and dehydration of this aldehyde via an aldol reaction; re-hydrogenation of the aldol product to a new alcohol (for example, butanol). This reaction sequence has been described in the prior art for a wide variety of alcohols; however, ethanol is known to be a particularly challenging starting material (Journal of Molecular Catalysis A: Chemical, Volume 200, Issues 1-2, 2003, Pages 137-146 with particular reference to section 3.1) and more efficient processes for the conversion of ethanol to butanol are currently unknown. The production of butanol from ethanol using iridium catalysts has been reported (Chemistry Letters, 2009, 38, page 838). However, the selectivity of the reaction is relatively poor with butanol accounting for only a small percentage of the products obtained.

There would be benefit in the development of a catalysed process for the conversion of ethanol to higher alcohols for example butanol which can achieve high selectivity to the desired alcohol or alcohols, and high conversion for example compared with alternative biosynthetic pathways.

Aspects of the present invention seek to mitigate or solve one or more of the problems indicated above and/or other problems.

According to an aspect of the invention there is provided a method for use in a process for the conversion of an alcohol into a product, the method including the step of contacting a composition comprising a first alcohol with a catalyst composition, in which the catalyst composition comprises:

i) a source of a Group VIII transition metal, wherein the Group VIII transition metal is selected from one or more of the group comprising Fe, Ru, Os;
ii) a phosphine ligand of formula PR¹R²R³, wherein R¹, R² and R³ are the same or different, and wherein one or more of R¹, R², and R³ include a heteroatom-substituted hydrocarbon group; and
iii) a base.

The phosphine ligand of formula PR¹R²R³ may be a multi-dentate ligand with respect to the Group VIII metal.

In some preferred examples of the invention, the heteroatom binds to the Group VIII metal. In some examples good conversion and/or selectivity to product alcohol can be achieved with the use of multi-dentate, for example, bi-dentate ligands. In some examples, the multi-dentate ligand may comprise a tri-dentate ligand. The catalyst may include two or more ligands, which may have the same, or different denticity from each other.

The heteroatom may comprise one or more selected from the group comprising O, S, N and P, preferably selected from the group comprising N and P.

In some preferred examples, the ligand comprises a bi-dentate P˜P (diphosphine) or P˜N ligand. In some examples, only one of R¹, R², and R³ include a heteroatom-substituted hydrocarbon group.

The phosphine ligand may have a formula selected from R₂P(CH₂)ₓPR₂ and R₂P{N(R)}ₓPR₂, where x is 0, 1, 2, 3, 4 or more.

The phosphine ligand may comprise a bi-dentate ligand with respect to the Group VIII metal. The phosphine ligand may comprise a diphosphine group —[P{link}P]— wherein a linking group {link} has a backbone including fewer than two atoms.

The phosphine ligand may have the formula R¹R²P{link}PR⁶R⁷, wherein R¹, R², R⁶, and R⁷ are the same or different, and {link} is a direct link between the phosphine groups, or {link} is a group comprising a single atom linking the phosphine groups.

The phosphine ligand may comprise a diphosphine ligand having a formula selected from R¹R²PCH₂PR⁶R⁷ and R¹R²PN(R)PR⁶R⁷, wherein R¹, R², R⁶, and R⁷ are the same or different. R¹ and R² may be the same and/or R⁶ and R⁷ may be the same.

R¹, R², R⁶ and R⁷ may be independently selected from hydrogen, hydrocarbon groups and heteroatom-substituted hydrocarbon groups. Two or more of the groups R¹, R², R⁶ or R⁷ may be linked, for example so that they form a cyclic structure together with the phosphorus atom.

The phosphine ligand may have a formula selected from R₂PCH₂PR₂ and R₂PN(R)PR₂.

R may be selected from hydrogen, hydrocarbon groups and heteroatom-substituted hydrocarbon groups. R may comprise for example Ph (phenyl group), Me (methyl), Et, (ethyl), Pr (propyl, iso or normal), tBu, (tertiary butyl), benzyl, tolyl (ortho, meta or para), anisyl (ortho, meta or para), or PhO (phenoxy).

The phosphine ligand may have the formula R¹R²P{link}NR⁶R⁷, wherein R¹, R², R⁶, and R⁷ are the same or different, and {link} is a direct link between the phosphine group and the nitrogen donor group. The phosphine ligand may comprise a P˜N ligand having a formula selected from R¹, R²P(CR₂)ₙNR⁶R⁷. R¹, R², R⁶, and R⁷ are the same or different and are for example as defined above. n is 1, 2, 3 or 4. R may be selected from hydrogen, hydrocarbon groups and heteroatom-substituted hydrocarbon groups. One R group may be linked to R⁶ in this embodiment, so that a nitrogen heterocycle is formed (and the other R group and R⁷ are made redundant).

In examples of the invention, the conversion is carried out at a relatively low hydrogen partial pressure.

It would have been considered to be a general requirement in processes for the conversion of an alcohol to a higher alcohol that the presence of hydrogen was required in the conversion step, for the conversion to proceed and for an appropriate selectivity to be achieved. For example, European Patent Application No. 2221289 describes that a partial hydrogen pressure of 0.1 MPa or higher is required for the conversion, with partial pressures of 0.5 to 20 MPa being described. The present inventors have, however, found surprisingly that conversion processes of aspects of the present invention using catalyst compositions described herein can proceed under conditions of lower hydrogen partial pressure.

In examples, the conversion process is carried out at a hydrogen partial pressure of less than 0.1 MPa.

This feature is of particular importance and is provided independently. Thus a further aspect of the invention provides a method for the conversion of an alcohol to a product, the method including the step of contacting a composition comprising a first alcohol with a catalyst composition, in which the catalyst composition comprises:

i) a source of a Group VIII transition metal, wherein the Group VIII transition metal is selected from one or more of the group comprising Fe, Ru, Os;
ii) a phosphine ligand of formula PR¹R²R³, wherein R¹, R² and R³ are the same or different; and
iii) a base.
wherein the conversion is carried out at a hydrogen partial pressure of less than 0.1 MPa.

For example, the hydrogen partial pressure may be less than 0.05 MPa. In some examples, the hydrogen partial pressure may be substantially 0 MPa.

This aspect of the invention finds application in relation to catalysts including mono-dentate phosphine ligands as well as to catalysts including multi-dentate ligands, for example where one or more of R¹, R², and R³ include a heteroatom-substituted hydrocarbon group.

According to an aspect of the invention there is provided a method for use in a process for the conversion of an alcohol into a product, the method including the step of contacting a composition comprising a first alcohol with a catalyst composition, in which the catalyst composition comprises:

i) a source of a Group VIII transition metal;
ii) a phosphine ligand of formula PR¹R²R³, wherein R¹, R² and R³ are the same or different; and
iii) a base.

In relation to this and/or other aspects of the invention described herein, preferably a first alcohol is contacted with the catalyst composition, and the product comprises a second alcohol.

In examples of the invention, the second alcohol is a higher alcohol than the first alcohol. Thus aspects of the invention described herein relate to a process for the conversion of an alcohol to a higher alcohol. In some examples of applications of aspects of the invention, the higher alcohol may represent the final product; in other examples, the alcohol may be subject to further conversion, to the product, for example to an alkene. The further conversion may be effected using the same or additional conversion catalyst, which may be present in combination with the catalyst, or may be physically separate. Similarly, the starting composition may be a pre-cursor to an alcohol in some examples, for example an acetaldehyde. Thus aspects of the invention may be directed to a conversion process including the conversion of an alcohol to a higher alcohol according to any aspect described herein.

In preferred examples of the invention, catalyst compositions described in the present invention convert a first alcohol to a second alcohol. In preferred examples, as discussed further below, the purity of the second alcohol generated as a product of the catalysed reaction is high, for example greater than 80% or 90%. Preferably the reaction products are such that substantially no further reaction and/or separation of the second alcohol from other reaction products is required.

In preferred examples, the conversion is highly selective giving a high yield of the second alcohol in favour of other products. In examples described below, selectivity of over 90% has been achieved. Thus while it is envisaged that the product including the second alcohol may include a mixture of two or more different alcohols, preferably the second alcohol is present in at least 80 wt % based on the weight of alcohols in the conversion products, and preferably at least 90 wt %.

Preferably the second alcohol has the general formula H(C₂H₄)ₙOH. Preferably n=2, 3 or 4. It is preferred in some examples that for at least 80 wt % of the second alcohol n=2.

Preferably at least 80 wt % of the products include an alcohol having a general formula H(C₂H₄)ₙOH, preferably wherein n=2.

In examples where butanol, for example butan-1-ol, is formed other products may include for example ethyl acetate, hexan-1-ol, 2-ethylbutan-1-ol, octan-1-ol and 2-ethylhexan-1-ol.

In some examples where butanol is formed as a product, the method may be selective to the production of one or more butanol. For example, the butanol produced may be substantially all butan-1-ol. In other example, substantially all the butanol may be butan-2-ol. There are other possibilities: for example, the selectivity may be less and two or more different butanols may be formed.

Preferably the conversion products include less than 10% branched hexanol. This is a product which may be preferentially formed in known methods attempting to convert ethanol to butanol.

In examples of the invention described, the alcohol to be converted comprises ethanol. For example, the method may be used to convert bio-ethanol, for example ethanol which is derived from biomass, for example produced by the fermentation of sugars. Other sources of ethanol may be used, from biomass and/or other sources.

The product preferably comprises a second alcohol, the second alcohol being a higher alcohol than the first, converted alcohol. In examples described, the product comprises butanol. Where reference is made herein to butanol, preferably unless apparent to the contrary the term includes isobutanol (or 2-methyl 1-propanol), 1-butanol and/or 2-butanol. In examples described herein, the butanol formed substantially, or completely consists of butan-1-ol.

Preferably the Group VIII transition metal is selected from one or more of the group comprising Fe, Ru, Os. In some preferred examples, the Group VIII transition metal comprises Ru.

The source of a group VIII metal can be the metal itself in some examples. The metal may for example be dispersed on a support material such as carbon, silica or alumina. Alternatively or in addition, the Group VIII metal may comprise a compound of the metal.

The Group VIII metal may comprise a component of a complex comprising a species of formula [M(L)ₙ]ₘ

where M is a Group VIII metal,

L is a ligand,

n is an integer from 1 to 8, and

m is an integer representing the nuclearity of the complex.

The complex may be charged. Preferably M of the complex is one or more of Fe, and Ru, preferably Ru.

The L groups may be the same or different and are ligands, for example chloride, bromide, iodide, hydride, alkoxide, amide, acetate, acetylacetonate, alkyl (for example methyl, ethyl, butyl), amine, ether, a hydrocarbon or substituted hydrocarbon ligand (for example η³-allyl, η⁴-butadiene, η⁵-cyclopentadienyl, η⁶-arene) water, CO, NO, phosphines (for example triphenylphosphine, trimethyl phosphine, trimesityl phosphine or triphenoxyphosphine), pyridine, alcohols, alkenes, alkynes, or N-heterocyclic carbenes. L groups may also be solid state materials that act as ligands and produce a supported metal species, for example silica, alumina, zeolites or poly(vinyl pyridine). Particularly preferred ligands include chloride, bromide, iodide, hydride, acetate, acetylacetonate, a hydrocarbon or substituted hydrocarbon ligand (for example η³-allyl, η⁴-butadiene, η⁵-cyclopentadienyl, η⁶-arene), water, CO, phosphines.

n is an integer from 1 to 8, preferably from 2 to 6. m is an integer representing the nuclearity of the complex, When m=1 the complex is a monomer, when m=2 the complex is a dimer, etc. and is generally from 1 to 8, preferably 1 or 2 although other values are possible. Generally the nature and number of the ligands L is selected to achieve suitable stability of the complex. When the overall complex has a charge, this will preferably be balanced by a suitable counter ion, for example Cl, Br, I, BF₄, PF₆, SbF₆.

A single source of a Group VIII transition metal or a mixture of two or more sources may be used. It is preferred in may examples that a single source be used. Suitable sources for Ru metal include for example

### RuCl₃.3H₂O

### RuI₃

### Ru(acetylacetonate)₃

### Ru(PPh₃)₃Cl₂

### Ru(PPh₃)₃(H)(CO)Cl

### [Ru(C₆H₆)Cl₂]₂

[Ru(arene)Cl₂]₂ (where arene is, for example, benzene or cymene)

### Ru(CO)₃Cl₂

### [Ru(CO)₄]₃

m is an integer for which the value is from 1 to 8, preferably 1 or 2.

Preferably in the phosphine ligand of formula PR¹R²R³, one or more, but preferably one, of R¹, R², and R³ include a heteroatom-substituted hydrocarbon group. The heteroatom may comprise one or more atoms selected from the group comprising O, S, N and P. As discussed further below, the heteroatom can in some examples also bind to the Group VIII metal, giving a bi-dentate or multi-dentate ligand. The heteroatom preferably comprises one or more atoms selected from the group N and P. The R¹, R², and/or R³ are preferably selected from one or more of the group comprising {C(R⁴)₂}ₓN(R⁵)₂, C(R⁴)ₓN(P⁵)₂, {N(R⁴)}_{y}N(P⁵)₂ where R⁴ and R⁵ are independently selected from H, hydrocarbon groups or heteroatom-substituted hydrocarbon groups.

The phosphine ligand of formula PR¹R²R³ is preferably a multi-dentate ligand with respect to the Group VIII metal. For example, the phosphine ligand is x-dentate, where x is greater than 1, for example where x is two (bidentate) or more, preferably three (tridentate) or more. Generally it is expected that x will be 4 or less.

In the phosphine ligand of formula PR¹R²R³, R¹, R², and R³ groups can be the same or different and are preferably H, hydrocarbon groups or heteroatom-substituted hydrocarbon groups. Two or more of the groups R¹, R², and R³ may also be linked so as to form a cyclic structure. Suitable hydrocarbon groups are linear, branched or cyclic alkyl groups with 1 to 50 carbon atoms, (for example: methyl, ethyl, n-propyl, i-propyl, butyl, pentyl, hexyl, octyl, decyl, cyclopentyl, cyclohexyl); or aryl or substituted aryl groups (for example phenyl, ortho-tolyl, meta-tolyl, para-tolyl, ethylphenyl, isopropylphenyl, t-butylphenyl, 2,6-dimethylphenyl, 2,4-dimethylphenyl, 3,5-dimethylphenyl, 2,6-diisopropylphenyl, 2,4,6-trimethylphenyl, 2,4,6-triisopropylphenyl, naphthyl, benzyl). Suitable heteroatom-substituted hydrocarbon groups may have one or more heteroatoms and may be bound via a carbon atom or heteroatom to the phosphorus, (for example CF₃, CF₂CF₃, CH₂OMe, CH₂NMe₂, fluorophenyl, perfluorophenyl, chlorophenyl, bromophenyl, C₆H₄(CF₃), C₆H₃(CF₃)₂, C₆H₄(OMe), C₆H₃(OMe)₂, C₆H₄(SO₃H), OMe, OEt, OPh, NMe₂, NEt₂, NPh₂).

A particular (and preferred) subset of heteroatom-substituted hydrocarbon groups contain a heteroatom which may bind to the Group VIII metal in addition to the phosphorus atom of the ligand to form a bidentate ligand. Suitable heteroatoms include O, S, N and P; N and P are preferred; P is particularly preferred. Examples of suitable heteroatom-substituted hydrocarbon groups of this type are {C(R⁴)₂}ₓN(R⁵)₂, C(R⁴)ₓN(P⁵)₂, {N(R⁴)}_{y}N(P⁵)₂ where R⁴ and R⁵ are independently selected from H, hydrocarbon groups or heteroatom-substituted hydrocarbon groups. Suitable hydrocarbon groups include those that are linear, branched or cyclic alkyl groups with 1 to 50 carbon atoms, (for example: methyl, ethyl, n-propyl, i-propyl, butyl, pentyl, hexyl, octyl, decyl, cyclopentyl, cyclohexyl); or aryl or substituted aryl groups (for example phenyl, ortho-tolyl, meta-tolyl, para-tolyl, ethylphenyl, fluorophenyl, perfluorophenyl, chlorophenyl, bromophenyl, C₆H₄(CF₃), C₆H₃(CF₃)₂, C₆H₄(OMe), C₆H₃(OMe)₂, isopropylphenyl, t-butylphenyl, 2,6-dimethylphenyl, 2,4-dimethylphenyl, 3,5-dimethylphenyl, 2,6-diisopropylphenyl, 2,4,6-trimethylphenyl, 2,4,6-triisopropylphenyl, naphthyl, benzyl). Suitable heteroatom-substituted hydrocarbon groups may have one or more heteroatoms (for example CF₃, CF₂CF₃, CH₂OMe, CH₂NMe₂, CH₂CH₂NH₂, fluorophenyl, perfluorophenyl, chlorophenyl, bromophenyl, C₆H₄(CF₃), C₆H₃(CF₃)₂, C₆H₄(OMe), C₆H₃(OMe)₂, C₆H₄(SO₃H), OMe, OEt, OPh, NMe₂, NEt₂, NPh₂). When there are plural R⁴ or R⁵ groups, they may be the same or different. Groups R⁴ and/or R⁵ may also be linked so as to form a cyclic structure. It is preferred that R⁴ is H or a hydrocarbon group with 1 to 6 atoms. x is preferably an integer between 1 and 6; preferably between 1 and 3. y is preferably 1 or 2. It can be seen that is more than one of the groups R¹, R², and R³ contain a heteroatom that may coordinate to the Group VIII metal then a tridentate or tetradentate ligand may be formed.

Examples of particularly preferred formulations for the phosphine ligand of formula PR¹R²R³ include

PPh₃

P(C₆H₁₁)₃

P(iPr)₃

P(tBu)₃

Ph₂PCH₂PPh₂

Ph₂PH(Me)PPh₂

(2-MeC₆H₄)₂PCH₂P(2-MeC₆H₄)₂

(2-iPrC₆H₄)₂PCH₂P(2-iPrC₆H₄)₂

(2-MeOC₆H₄)₂PCH₂P(2-MeOC₆H₄)₂

(iPr)₂PCH₂P(iPr)₂

(C₆H₁₁)₂PCH₂P(C₆H₁₁)₂

(tBu)₂PCH₂P(tBu)₂

(PhO)₂PCH₂P(OPh)₂

Ph₂PCH₂CH₂PPh₂

Ph₂PCH₂CH₂CH₂PPh₂

Ph₂PCH₂CH₂CH₂CH₂PPh₂

1,2-Ph₂PC₆H₄PPh₂

Ph₂PN(H)PPh₂

Ph₂PN(Me)PPh₂

Ph₂PN(iPr)PPh₂

Ph₂PN(Ph)PPh₂

(2-MeOC₆H₄)₂PN(Me)P(2-MeOC₆H₄)₂

(2-MeC₆H₄)₂PN(Me)P(2-MeC₆H₄)₂

(2-iPrC₆H₄)₂PN(Me)P(2-iPrC₆H₄)₂

Ph₂PN(Me)N(Me)PPh₂

Ph₂PCH₂CH₂NH₂

Ph₂PCH₂CH₂NMe₂

Ph₂PCH₂CH₂OH

Ph₂PCH₂CH₂OMe

Ph₂PCH₂CH₂SH

Ph₂PCH₂CH₂SMe

Ph₂PCH₂CH₂P(Ph)CH₂CH₂PPh₂

Ph₂PCH₂CH₂N(H)CH₂CH₂PPh₂

Ph₂PCH₂CH₂OCH₂CH₂PPh₂

CH₃C(CH₂PPh₂)₃

Preferably the base's conjugate acid has a pKa greater than 5. The base may comprise any compound with a conjugate acid having a pKa preferably greater than 5. Suitable bases include metal alkoxides (such as NaOMe, KOMe, NaOEt, NaOBu), metal hydroxides, amines and metal carbonates, or basic solid-state materials. It is preferred that the base is a metal alkoxide; more preferred are sodium or potassium methoxide, ethoxide and butoxide. These may also be formed in the reaction media by reaction of sodium or potassium metal with an alcohol.

The molar ratio between the source of a Group VIII transition metal and phosphine ligand of formula PR¹R²R³ is preferably between 1:10 and 10:1; preferably the ratio is 1:2 to 1:1. The molar ratio between the source of a Group VIII metal and the base is preferably between 1:1 and 1:1000; preferably between 1:1 and 1:100.

The source of a Group VIII transition metal, phosphine ligand of formula PR¹R²R³ and base may be added in any order, separately or together in the process reaction.

They may also be pre-reacted to synthesise a pre-formed Group VIII transition metal-phosphine ligand complex by various methods known to those skilled in the art.

The method may further include the step of reacting the Group VIII transition metal and phosphine ligand to form a catalyst complex prior to conversion of the alcohol.

These typically involve reacting the source of a Group VIII with the phosphine ligand in a suitable solvent. This pre-formed Group VIII transition metal-phosphine ligand complex may then be added to the process reaction. In a similar way, the source of a Group VIII transition metal and/or phosphine ligand or pre-formed Group VIII transition metal-phosphine ligand complex may be pre-reacted with the base. Examples of such pre-formed complexes in which Ph₂PCH₂PPh₂ is used as an exemplar diphosphine ligand include: [Ru(η⁶-cymene)(Ph₂PCH₂PPh₂)Cl]Cl; [Ru(η⁶-cymene)(Ph₂PCH₂PPh₂)Cl]PF₆; [Ru(η⁶-cymene)(Ph₂PCH₂PPh₂)H]PF₆; [Ru(η⁵-C₅H₅)(Ph₂PCH₂PPh₂)Cl]; [Ru(Ph₂PCH₂PPh₂)(Cl)(μ-Cl)]₂; [Ru(Ph₂PCH₂PPh₂)₂Cl₂]. In other examples, other ligands described herein could be substituted for the Ph₂PCH₂PPh₂ ligand.

In the process reactor, the catalyst may be in the same phase as the ethanol (a homogeneous reaction in the liquid phase) or in a different phase (for example a solid catalyst and liquid or gaseous ethanol). The amount of catalyst in the process reactor (based on molar ratio ethanol to the Group VIII transition metal) will vary depending on the specific reactor configuration used but will typically be between 10⁸ and 100 to 1. In a homogeneous reaction, the ethanol or butanol product may also act as a diluent, or an additional diluent solvent may be added. Suitable diluents include alkanes (such as butane, pentane, hexane, cyclohexane), arenes (such as benzene, toluene, xylene), alcohols (such as methanol, ethanol, propanol, butanol), ethers (such as diethyl ether, dibutyl ether, tetrahydrofuran), carboxylic acids and carboxylic acid derivatives (such as acetic acid, ethyl acetate), water, or mixture thereof. The reaction temperature may be between 0° C. and 500° C.; preferably between 70° C. and 200° C. The process reaction may be performed under a pressure of hydrogen.

The ethanol used in the reaction may be derived from fossil fuel resources (typically via hydration of ethylene) or, preferably, from biosynthetic conversion (fermentation) of biomass.

The method may further include the step of separating a second alcohol from the reaction composition.

Also provided by the invention is a method for the conversion of a first alcohol to a second alcohol, the second alcohol being a higher alcohol than the first, the method including the steps of:
i) contacting a composition comprising the first alcohol with a catalyst composition according to a method described herein; and
ii) separating the second alcohol from the components of the catalyst reaction.

The separation may be carried out by any appropriate method, for example by passing the reaction product to a membrane separator. As discussed above, preferably the butanol is sufficiently pure that it may be used at least for some applications without further purity being required.

In examples described, the first alcohol comprises ethanol. While aspects of the invention find particular application in relation to the conversion of ethanol, it is envisaged that catalysts described herein may be used in the conversion of other components, for example other alcohols. For example, the first alcohol may include one or more of butanol, methanol and glycerol. The second alcohol preferably comprises butanol for some applications.

Also provided by a further aspect of the invention is a method of converting a first alcohol to a second alcohol, the method including the step of contacting a composition comprising a first alcohol with a catalyst composition, in which the catalyst composition comprises:

i) a source of a Group VIII transition metal;
ii) a phosphine ligand of formula PR¹R²R³, wherein R¹, R² and R³ are the same or different;
wherein the purity of the second alcohol is at least 80%. The purity may be determined for example by Gas Chromatography.

Aspects of the invention find particular application in relation to the production of butanol, but it is envisaged that catalysts described herein may be used in the production of other products, for example higher alcohols.

Also provided by the invention is butanol obtainable by a method described herein.

The invention also provides a method of converting a first alcohol to a second alcohol, the method including the step of contacting a composition comprising a first alcohol with a catalyst composition, in which the catalyst composition comprises:

i) a source of a Group VIII transition metal, wherein the Group VIII transition metal is selected from one or more of the group comprising Fe, Ru, Os;
ii) a phosphine ligand of formula PR¹R²R³, wherein R¹, R² and R³ are the same or different, and wherein one or more of R¹, R², and R³ include a heteroatom-substituted hydrocarbon group;
wherein the purity of the second alcohol is at least 80%.

Also provided by an aspect of the invention a catalyst composition including

i) a source of a Group VIII transition metal, wherein the Group VIII transition metal is selected from one or more of the group comprising Fe, Ru, Os;
ii) a phosphine ligand of formula PR¹R²R³, wherein R¹, R² and R³ are the same or different, and wherein one or more of R¹, R², and R³ include a heteroatom-substituted hydrocarbon group; and
iii) a base.

The Group VIII metal may comprise Ru.

The phosphine ligand may form a multi-dentate ligand complex with the Group VIII transition metal. The phosphine ligand may form a bi-dentate ligand with respect to the Group VIII metal.

The heteroatom preferably comprises one or more selected from the group comprising O, S, N and P, preferably selected from the group comprising N and P.

In some examples, only one of R¹, R², and R³ include a heteroatom-substituted hydrocarbon group.

The phosphine ligand may have the formula R¹R²P{link}PR⁶R⁷, wherein R¹, R², R⁶, and R⁷ are the same or different, and {link} is a direct link between the phosphine groups, or {link} is a group comprising a single atom linking the phosphine groups.

The phosphine ligand may comprise a diphosphine ligand having a formula selected from R¹R²PCH₂PR⁶R⁷ and R¹R²PN(R)PR⁶R⁷ wherein R¹, R², R⁶, and R⁷ are the same or different.

R¹ and R² may be the same and/or R⁶ and R⁷ may be the same.

R¹, R², R⁶ and R⁷ may be independently selected from hydrogen, hydrocarbon groups and heteroatom-substituted hydrocarbon groups.

The phosphine ligand may have a formula selected from R₂PCH₂PR₂ and R₂PN(R)PR₂.

R may be selected from hydrogen, hydrocarbon groups and heteroatom-substituted hydrocarbon groups. R may comprise for example Ph (phenyl group), Me (methyl), Et, (ethyl), Pr (propyl, iso or normal), tBu, (tertiary butyl), benzyl, tolyl (ortho, meta or para), anisyl (ortho, meta or para), or PhO (phenoxy).

The phosphine ligand may have the formula R¹R²P{link}NR⁶R⁷, wherein R¹, R², R⁶, and R⁷ are the same or different, and {link} is a direct link between the phosphine group and the nitrogen donor group. The phosphine ligand may comprise a P˜N ligand having a formula selected from R¹R²P(CR₂)ₙNR⁶R⁷. R¹, R², R⁶, and R⁷ are the same or different and are for example as defined above. n is 1, 2, 3 or 4. R may be selected from hydrogen, hydrocarbon groups and heteroatom-substituted hydrocarbon groups. One R group may be linked to R⁶ in this embodiment, so that a nitrogen heterocycle is formed (and the other R group and R⁷ are made redundant).

Where the catalyst includes more than one ligand, the ligands may be the same or different from each other. More than one of the ligands may be a phosphine ligand as described herein in relation to any aspect of the invention.

The invention thus also provides catalysts which can be used for example in the methods described herein. The catalyst may include one or more of the features described herein.

Also provided by the invention is a catalyst composition, for example for use in any of the methods described herein.

According to a further aspect of the invention, there is provided a catalyst composition including

i) a source of a Group VIII transition metal;
ii) a phosphine ligand of formula PR¹R²R³, wherein R¹, R² and R³ are the same or different; and
iii) a base.

The composition may further include additional features of the catalyst composition described herein.

Preferably the phosphine ligand forms a multi-dentate ligand complex with the Group VIII transition metal. As discussed herein, the phosphine ligand may be bi-, tri-dentate or even more.

R¹, R² and/or R³ preferably include a hetero-atom substituted hydrocarbon. R¹, R² and/or R³ may include a hetero-atom substituted hydrocarbon group, wherein the heteroatom is selected from N and P.

Also provided by the invention is the use of a catalyst composition described herein in the conversion of alcohols.

Aspects of the invention also provide a method or product being substantially as herein described.

Also provided by the invention is the use of a catalyst in the conversion of ethanol to butanol with a conversion rate over 80%, preferably over 90%. Preferably the conversion of ethanol to 1-hexanol is less than 20%, preferably less than 10%.

The invention also provides a method, catalyst, or apparatus being substantially as herein described, optionally having reference to the figure. Features described herein may be combined in any appropriate combination. Features of one aspect of the invention may be applied to other aspects as appropriate. For example method features may be applied to product, apparatus or composition aspects and vice versa.

Features of embodiments of the invention will now be described, purely by way of example having reference to the figures in which:

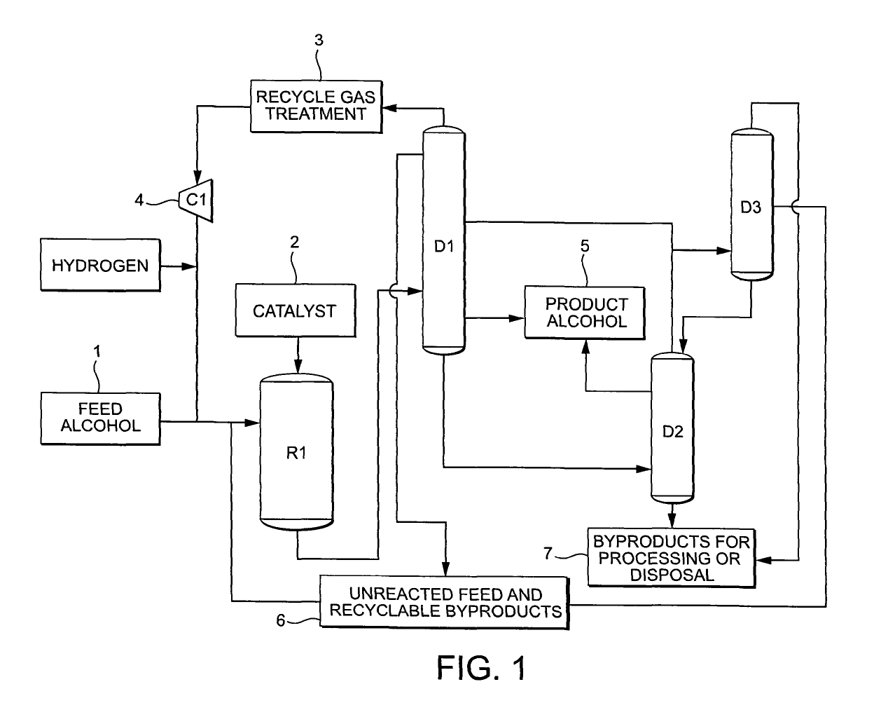

FIG. 1 is a schematic illustration of an example industrial process for converting a lower alcohol to a higher alcohol.

### EXAMPLES

For all of the examples, the procedure was performed under inert atmosphere (Argon or Nitrogen) using degassed and dried reagent-grade solvents unless otherwise stated. Catalyst-screening experiments were performed using a Radleys Carousel 12 Reaction Station (Classic). Product analysis was performed on a Varian Saturn 2100T GC/MS using a FactorFour capillary column VF-5 ms. NMR analysis was performed on an ECP 300 MHz spectrometer.

### General Procedure for Examples 1 to 21

A Group VIII metal source (0.1 mol %), phosphine ligand (0.1 mol %) (or 0.1 mol % of a pre-formed metal-ligand complex) and solid base (5 mol %) were added to a screw-top glass vial for a Radleys 12-cell reaction vessel or a 100 ml volume Parr stainless steel autoclave with stir-bar under inert atmosphere. Using standard Schlenk-line techniques, 5 mL (85.63 mmol) Ethanol was then added with rigorous stirring at room temperature. The reaction vessel was then sealed and mixtures were then heated to 150° C. with continued stirring for 4 hours. During this time, the internal pressure of the reaction vessel increased so that in each case the final pressure was between 0.1 and 1 MPa. The run was terminated by cooling and release of pressure.

Conversion is stated as a percentage of maximum possible butanol yield (42.815 mmol) based on the ethanol added. GC/MS analysis was carried out using methanol as a diluent and was calibrated using mesitylene as a standard.

### Example 1

(Cymene)ruthenium dichloride dimer (0.05 mol %), Bis(diphenylphosphino)methane (0.1 mol %) and sodium ethoxide (5 mol %) were stirred with 5 mL Ethanol (85.63 mmol) for 4 hours at 150° C. Reaction mixture was filtered through Celite (RTM Celite Corporation, US) and analysed by GC/MS. GC-trace showed 24.1% conversion to butanol, >99% selectivity. The butanol formed was substantially 100% butan-1-ol. The selectivity is determined for example by integration of GC signals for butanol compared with all of the other components in the product other than ethanol.

### Example 2

(Cymene)ruthenium dichloride dimer (0.05 mol %), 1,2-Bis(diphenylphosphino)ethane (0.1 mol %) and sodium ethoxide (5 mol %) were stirred with 5 mL Ethanol (85.63 mmol) for 4 hours at 150° C. Reaction mixture was filtered through Celite and analysed by GC/MS. GC-trace showed 13.2% conversion, 97% selectivity.

### Example 3

(Cymene)ruthenium dichloride dimer (0.05 mol %), 1,3-Bis(diphenylphosphino)propane (0.1 mol %) and sodium ethoxide (5 mol %) were stirred with 5 mL Ethanol (85.63 mmol) for 4 hours at 150° C. Reaction mixture was filtered through Celite and analysed by GC/MS. GC-trace showed 14.8% conversion, >99% selectivity.

### Example 4

(Cymene)ruthenium dichloride dimer (0.05 mol %), 1,3-Bis(diphenylphosphino)propane (0.1 mol %), 1,7-octadiene (10 mol %) and sodium ethoxide (5 mol %) were stirred with 5 mL Ethanol (85.63 mmol) for 4 hours at 150° C. Reaction mixture was filtered through Celite and analysed by GC/MS. GC-trace showed 15.2% conversion, >99% selectivity.

### Example 5

(Cymene)ruthenium dichloride dimer (0.05 mol %), N-methyl-Bis(diphenylphosphino)amine (0.1 mol %) and sodium ethoxide (5 mol %) were stirred with 5 mL Ethanol (85.63 mmol) for 4 hours at 150° C. Reaction mixture was filtered through Celite and analysed by GC/MS. GC-trace showed 10.5% conversion, 80% selectivity.

### Example 6

(Cymene)ruthenium dichloride dimer (0.05 mol %), 1,3-Bis(diphenylphosphino)propane (0.1 mol %) and sodium hydroxide (5 mol %) were stirred with 5 mL Ethanol (85.63 mmol) for 4 hours at 150° C. Reaction mixture was filtered through Celite and analysed by GC/MS. GC-trace showed 0.9% conversion, 95% selectivity.

### Example 7

(Cymene)ruthenium dichloride dimer (0.05 mol %), 1,3-Bis(diphenylphosphino)propane (0.1 mol %) and potassium hydroxide (5 mol %) were stirred with 5 mL Ethanol (85.63 mmol) for 4 hours at 150° C. Reaction mixture was filtered through Celite and analysed by GC/MS. GC-trace showed 8.9% conversion, >99% selectivity.

### Example 8

(Cymene)ruthenium dichloride dimer (0.05 mol %), 1,3-Bis(diphenylphosphino)propane (0.1 mol %) and Caesium carbonate (5 mol %) were stirred with 5 mL Ethanol (85.63 mmol) for 4 hours at 150° C. Reaction mixture was filtered through Celite and analysed by GC/MS. GC-trace showed 9.0% conversion, >99% selectivity.

### Example 9

2-methylallyl)(1,5-cyclooctadiene)ruthenium (0.1 mol %), Bis(diphenylphosphino)methane (0.1 mol %) and sodium ethoxide (5 mol %) were stirred with 5 mL Ethanol (85.63 mmol) for 4 hours at 150° C. Reaction mixture was filtered through Celite and analysed by GC/MS. GC-trace showed 14.2% conversion, >99% selectivity

### Example 10

RuCl₃ hydrated crystals (0.1 mol %), Bis(diphenylphosphino)methane (0.1 mol %) and sodium ethoxide (5 mol %) were stirred with 5 mL Ethanol (85.63 mmol) for 4 hours at 150° C. Reaction mixture was filtered through Celite and analysed by GC/MS. GC-trace showed 24.6% conversion, >99% selectivity.

### Catalysts

The synthesis of some of the pre-formed catalysts, for example catalyst complexes that may be used in examples of aspects of the invention are now described purely by way of example.

### Synthesis of Pre-Formed Catalysts

### Synthesis 1

### Preparation of Chloro(p-cymene)(η²-diphenylphosphinomethane-κ²P)ruthenium

[RuCl₂(cymene)]₂ was added as a suspension in ethanol to a solution of 1.1 equivalents of the bis(diphenylphosphino)methane, also in ethanol. The mixture was then stirred at room temperature for 16 hours before the volume of the reaction was reduced by half in vacuo, and then chilled for 16 hours at −20° C. The mixture is then filtered to remove the precipitated excess phosphine and triturated with diethyl ether.

The analysis by ¹H and ³¹P NMR of the product was as follows:

¹H NMR (CDCl₃): δ7.68-7.41 (m, 20H), δ6.42 (d, 2H, J=6.23 Hz), δ6.34 (d, 2H, J=6.23 Hz) δ4.99 (dt, 1H, J¹=15.0 Hz, J²=10.3 Hz) δ4.57 (dt, 1H, J¹=15.0 Hz J²=12.82 Hz), δ2.51 (septet, 1H, J=6.96 Hz), δ1.57 (s, 3H), δ1.08 (d, 6H, J=6.96 Hz).

³¹P{¹H}NMR (CD3OD): δ 2.9 (s)

### Synthesis 2

### Preparation of Chloro(p-cymene)(η²-N-methyl-diphenylphosphinoamine-κ²P)ruthenium

[RuCl₂(cymene)]₂ was added as a suspension in ethanol to a solution of 1.1 equivalents of the N-methyl-bis(diphenylphosphino)amine, also in ethanol. The mixture was refluxed for 16 hours before the volume of the reaction was reduced by half in vacuo, and then chilled for 16 hours at −20° C. The mixture would then be filtered to remove the precipitated excess phosphine. No further purification was necessary, compound was spectrosopically pure. Product was analysed by ¹H and ³¹P NMR.

### Synthesis 3

### Preparation of Dichloro(p-cymene)(triphenylphosphine)ruthenium

[RuCl₂(cymene)]₂ was be added as a suspension in ethanol to a solution of 7 equivalents of the triphenylphosphine, also in ethanol. The mixture would be refluxed for 16 hours before the volume of the reaction was reduced by half in vacuo, filtered to remove excess phosphine and then chilled for 16 hours at −20° C. The product crystallized out of the cold ethanol and was washed with hexane.

The product was analysed by ¹H NMR (CDCl₃): δ7.83 (m, 6H, PPh₃), δ7.37 (m, 9H, PPh₃), δ5.20 (d, 2H, J=6.23 Hz), δ4.99 (d, 2H, J=6.23 Hz), δ2.86 (septet, 1H, J=6.96 Hz), δ1.87 (s, 3H), δ1.10 (d, 6H, J=6.96 Hz). ³¹P NMR (CDCl₃) δ24.8 (s)

### Synthesis 4

### Preparation of [RuCl₂(bis(diphenylphosphino)methane)₂]

To a solution of bis(diphenylphosphino)methane (2.5 eq) in ethanol, a solution of RuCl₃.xH₂O in water was added slowly, with stirring. The reaction mixture was refluxed for 3 hours, allowed to cool to room temperature and then diluted with water until a yellow precipitate appeared. The precipitate was filtered, washed with water and hexane, and dried under vacuum to give a yellow solid; ³¹P NMR (162 MHz, CDCl₃) δ −7.17; ¹H NMR (400 MHz, CDCl₃) δ 7.41-7.14 (m, 40H, aromatic CH), 5.06 (t, 2H, J=4.16 Hz, CH₂)

### Examples Using Pre-Formed Catalysts

### Example 11

Chloro(p-cymene)(η²-diphenylphosphinomethane-κ²P)ruthenium (0.1 mol %) and sodium ethoxide (5 mol %) were stirred with 5 mL Ethanol (85.63 mmol) for 4 hours at 150° C. The reaction mixture was filtered through Celite and analysed by GC/MS. GC-trace showed 44.7% conversion, >99% selectivity.

### Example 12

Chloro(p-cymene)(η²-diphenylphosphinomethane-κ²P)ruthenium (0.1 mol %) and sodium ethoxide (10 mol %) were stirred with 5 mL Ethanol (85.63 mmol) for 4 hours at 150° C. Reaction mixture was filtered through Celite and analysed by GC/MS. GC-trace showed 50% conversion, >99% selectivity.

### Example 13

Chloro(p-cymene)(η²-N-methyl-diphenylphosphinoamine-κ²P)ruthenium and sodium ethoxide (5 mol %) were stirred with 5 mL Ethanol (85.63 mmol) for 4 hours at 150° C. Reaction mixture was filtered through Celite and analysed by GC/MS. GC-trace showed 28.6% conversion, >99% selectivity.

### Example 14

[RuCl₂(bis(diphenylphosphino)methane)₂] and sodium ethoxide (5 mol %) were stirred with 5 mL Ethanol (85.63 mmol) for 240 hours at 150° C. Reaction mixture was filtered through Celite and analysed by GC/MS. GC-trace showed 10% conversion, 94% selectivity,

### Examples 15 to 18

Examples 15 to 18 are tabulated below. The same method as described in example 1 was followed, only with the indicated N—P ligand in place of a bidentate phosphine ligand.

| | | Conversion | Selectivity % |
|---|---|---|---|
| Example | Catalyst | (%) | butanol |
| 15 | 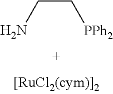 | 22 | 90 |
| 16 | 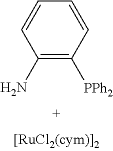 | 16 | 87 |
| 17 | 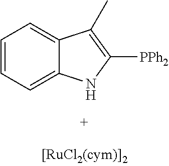 | 28 | 91 |
| 18 | 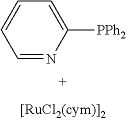 | 13 | 86 |

### Examples 19 and 20

Examples 19 and 20 are tabulated below. The same method as described in Example 1 was followed, only with the indicated tridentate P—P—P ligand in place of a bidentate phosphine ligand.

| | | Conversion | Selectivity % |
|---|---|---|---|
| Example | Catalyst | (%) | butanol |
| 19 | 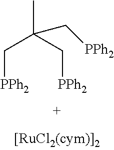 | 5 | 88 |
| 20 | 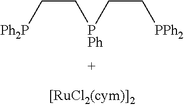 | 9 | 89 |

### Examples at Lower Pressure

### Examples 21 to 24

General procedure for Examples 21 to 24: These examples were performed using the same general method of Example 11, only the reactions were performed in an open system consisting round-bottom flask equipped with a reflux condenser at reflux temperature. The hydrogen pressure throughout the run was less than 0.1 MPa and essentially zero. These examples show that excellent selectivity and good conversion may be obtained at low hydrogen partial pressures (compare example 11 to 21, 13 to 22, and 23 to 24).

| | | Conversion | Selectivity % |
|---|---|---|---|
| Example | Catalyst | (%) | butanol |
| 21 | 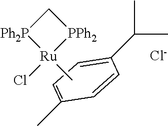 | 20 | 90 |
| 22 | 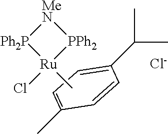 | 21 | 90 |
| 23 | Dichloro(p- | 4 | 95 |
| | cymene)(triphenylphosphine)ruthenium | | |

### Example 24

### Comparative Example to Example 23 at Higher Pressure

Using the same method as example 11. Dichloro(p-cymene)(triphenylphosphine)ruthenium and sodium ethoxide (5 mol %) were stirred with 5 mL Ethanol (85.63 mmol) for 4 hours at 150° C. A final pressure of 0.4 MPa was recorded. Reaction mixture was filtered through Celite and analysed by GC/MS. GC-trace showed 4.1% conversion, >99% selectivity.

### Example Industrial Process for Converting a Lower Alcohol to a Higher Alcohol

A commercial process to convert a lower alcohol to a higher alcohol using aspects of the present invention may be realised in many different ways, for example depending on the preferred conditions for the alcohol to be converted. By way of example, an industrial process employing features of the present invention may include some or all of the following steps:

### 1 Catalyst Preparation

### 2 Feed Pretreatment

### 3 Reaction

### 4 Primary Separation

### 5 Gas Treatment and Recycle

### 6 Product Refining

### 1. Catalyst Preparation

Catalyst is prepared when required either offsite by specialist suppliers or onsite, depending on the type of reaction system and the ease of catalyst preparation, transportation and storage.

### 2. Alcohol Feed Pretreatment

The alcohol to be converted may be pretreated to achieve the preferred conditions for reaction, which will vary according to the alcohol which is to be converted. Pretreatment may include the removal of trace contaminants deleterious to the process, and/or heating/cooling to preferred reaction conditions (phase, temperature and pressure).

### 3. Reaction [R1]

The feed alcohol 1 and the prepared catalyst 2 are fed to a reactor R1. In the reaction section, the alcohol to be converted is intimately contacted with the catalyst of this invention, which may be in solid or liquid form. Additional chemicals may be added to promote or moderate the reaction, along with hydrogen and/or other gas to maintain the required pressure. The reactor design will be appropriate to the form of the catalyst, the phase of the reaction and the need to add or remove heat. Lower alcohols may prefer vapour phase operation or require higher pressure to remain in liquid phase at reaction temperature, while higher alcohols may prefer liquid phase operation or require lower pressure operation to enable vaporisation at reaction temperature. Intimate contacting of the reactant and catalyst may be effected naturally (as with the flow of vapour phase reactant through a solid catalyst bed), mechanically by use of an agitator or hydraulically by liquid or vapour jet mixing. The reaction may be batch, semi-batch or continuous.

### 4. Primary Separation [D1]

The material leaving the reactor undergoes a primary separation in a primary separator D1 to separate the required product(s) from unreacted feed materials which can then be recycled to the reactor R1. This primary separation can take a number of forms, including flash separation, distillation, membranes or a combination of suitable techniques. Primary separation may also be combined with reaction in a reactive distillation system.

### 5. Gas Treatment and Recycle

Any unreacted feed materials in gas or vapour phase leaving the primary separation may be treated to remove trace contaminants deleterious to the process and/or heated/cooled and/or compressed/pumped to the appropriate conditions for recycle to the reactor. The example in FIG. 1 includes a recycle gas treatment unit 3 and a compressor 4.

### 6. Product Refining [D2 & D3]

The liquid phase materials leaving the primary separator D1 may be further refined to give for example

the desired product(s) at the required specification(s) for sale or use (Product 5)

unreacted materials and/or byproducts 6 for recycle to the reactor R1

non-recyclable byproducts 7 for further processing or disposal

Features of aspects of the invention have been described above by way of example only and variations may be made within the scope of the invention. For example, the filter medium may include any other appropriate material, for example another diatomaceous earth material and/or other filter material.

1. A method for use in a process for the conversion of an alcohol into a product, the method including the step of contacting a composition comprising a first alcohol with a catalyst composition, in which the catalyst composition comprises:
i) a source of a Group VIII transition metal, wherein the Group VIII transition metal is selected from one or more of the group comprising Fe, Ru, Os;
ii) a phosphine ligand of formula PR¹, R²R³, wherein R¹, R² and R³ are the same or different, and wherein one or more of R¹, R², and R³ include a heteroatom-substituted hydrocarbon group; and
iii) abase.

2. A method according to claim 1, wherein the phosphine ligand of formula PR¹, R²R³ is a multi-dentate ligand with respect to the Group VIII metal.

3. A method according to claim 1, wherein the heteroatom comprises one or more selected from the group comprising O, S, N and P.

4. A method according to claim 3, wherein the heteroatom comprises one or more selected from the group comprising N and P.

5. A method according to claim 1, wherein only one of R¹, R², and R³ include a heteroatom-substituted hydrocarbon group.

6. A method according to claim 1, wherein the phosphine ligand comprises a bi-dentate ligand with respect to the Group VIII metal.

7. A method according to claim 1, wherein the phosphine ligand comprises a diphosphine group —[P{link}P]— wherein a linking group {link} has a backbone including fewer than two atoms.

8. A method according to claim 1, wherein the phosphine ligand has the formula R¹, R²P{link}PR⁶R⁷, wherein R¹, R², R⁶, and R⁷ are the same or different, and {link} is a direct link between the phosphine groups, or {link} is a group comprising a single atom linking the phosphine groups.

9. A method according to claim 1, wherein the phosphine ligand comprises a diphosphine ligand having a formula selected from R¹, R²PCH₂PR⁶R⁷ and R¹, R²PN(R)PR⁶R⁷, wherein R¹, R², R⁶, and R⁷ are the same or different.

10. A method according to claim 8, wherein R¹ and R² are the same and/or R⁶ and R⁷ are the same.

11. A method according to claim 8, wherein R¹, R², R⁶ and R⁷ are independently selected from hydrogen, hydrocarbon groups and heteroatom-substituted hydrocarbon groups.

12. A method according to claim 1, wherein the phosphine ligand has a formula selected from R₂PCH₂PR₂ and R₂PN(R)PR₂.

13. A method according to claim 12, wherein R is selected from hydrogen, hydrocarbon groups and heteroatom-substituted hydrocarbon groups.

14. A method according to claim 13, wherein R is selected from Ph (phenyl), Me (methyl), Et, (ethyl), Pr (propyl, iso or normal), tBu, (tertiary butyl), benzyl, tolyl (ortho, meta or para), anisyl (ortho, meta or para), PhO (phenoxy).

15. A method according to claim 1 wherein the catalyst comprises a phosphine ligand having the formula R¹ R²P{link}NR⁶R⁷, wherein R¹, R², R⁶, and R⁷ are the same or different, and {link} is a direct link between the phosphine group and the nitrogen donor group.

16. A method according to claim 1 wherein the catalyst comprises a phosphine ligand comprising a P˜N ligand having a formula selected from R¹, R²P(CR₂)ₙNR⁶R⁷, wherein R¹, R², R⁶, and R⁷ are the same or different and wherein n is 1, 2, 3 or 4.

17. A method according to claim 15, wherein R¹ and R² are the same and/or R⁶ and R⁷ are the same.

18. A method according to claim 8, wherein R¹, R², R⁶ and R⁷ are independently selected from hydrogen, hydrocarbon groups and heteroatom-substituted hydrocarbon groups.

19. A method according to claim 16, wherein R is selected from hydrogen, hydrocarbon groups and heteroatom-substituted hydrocarbon groups.

20. A method according to claim 1 wherein the conversion process is carried out at a hydrogen partial pressure of less than 0.1 MPa.

21. A method for the conversion of an alcohol to a product, the method including the step of contacting a composition comprising a first alcohol with a catalyst composition, in which the catalyst composition comprises:
i) a source of a Group VIII transition metal, wherein the Group VIII transition metal is selected from one or more of the group comprising Fe, Ru, Os;
ii) a phosphine ligand of formula PR¹, R²R³, wherein R¹, R² and R³ are the same or different; and
iii) a base.
wherein the conversion is carried out at a hydrogen partial pressure of less than 0.1 MPa.

22. A method according to claim 20, wherein the hydrogen partial pressure is less than 0.05 MPa.

23. A method according to claim 20, wherein the ligand is mono-dentate with respect to the Group VIII metal.

24. A method according to claim 20, including a ligand which is multi-dentate with respect to the Group VIII metal.

25. A method according to claim 1, wherein a first alcohol is contacted with the catalyst composition, and the product comprises a second alcohol, the second alcohol preferably being a higher alcohol than the first, converted alcohol.

26. A method according to claim 1, wherein the second alcohol has the general formula H(C₂H₄)ₙOH, and preferably wherein n=2, 3 or 4.

27. A method according to claim 1, wherein at least 80 wt % of the products include an alcohol having a general formula H(C₂H₄)ₙOH, preferably wherein n=2.

28. A method according to claim 1, wherein the conversion products include less than 10% branched hexanol.

29. A method according to claim 1, wherein the first alcohol comprises ethanol and/or wherein the product comprises butanol.

30. A method according to claim 1, wherein the Group VIII transition metal comprises Ru.

31. A method according to claim 1, wherein the Group VIII metal is a component of a complex comprising a species of formula
[M(L)ₙ]ₘ
where M is a Group VIII metal selected from one or more of the group comprising Fe, Ru, Os,
L is a ligand,
n is an integer from 1 to 8, and
m is an integer representing the nuclearity of the complex, and preferably wherein m is an integer from 1 to 8, preferably 1 or 2.

32. A method according to claim 1, wherein the R¹, R², and/or R³ are selected from one or more of the group comprising {C(R⁴)₂}ₓN(R⁵)₂, C(R⁴)ₓN(P⁵)₂, {N(R⁴)}_{y}N(P⁵)₂ where R⁴ and R⁵ are independently selected from H, hydrocarbon groups or heteroatom-substituted hydrocarbon groups.

33. A method according to claim 1, wherein the base has a pKa greater than 5.

34. A method according to claim 1, further including the step of reacting the Group VIII transition metal and phosphine ligand to form a catalyst complex prior to conversion of the alcohol.

35. A method of converting a first alcohol to a second alcohol, the method including the step of contacting a composition comprising a first alcohol with a catalyst composition, in which the catalyst composition comprises:
i) a source of a Group VIII transition metal, wherein the Group VIII transition metal is selected from one or more of the group comprising Fe, Ru, Os;
ii) a phosphine ligand of formula PR¹R²R³, wherein R¹, R² and R³ are the same or different, and wherein one or more of R¹, R², and R³ include a heteroatom-substituted hydrocarbon group;
wherein the purity of the second alcohol is at least 80%.

36. A catalyst composition including
i) a source of a Group VIII transition metal, wherein the Group VIII transition metal is selected from one or more of the group comprising Fe, Ru, Os;
ii) a phosphine ligand of formula PR¹, R²R³, wherein R¹, R² and R³ are the same or different, and wherein one or more of R¹, R², and R³ include a heteroatom-substituted hydrocarbon group; and
iii) a base.

37. A catalyst composition according to claim 36, wherein the Group VIII metal comprises Ru.

38. A catalyst composition according to claim 36, wherein the phosphine ligand forms a multi-dentate ligand complex with the Group VIII transition metal.

39. A catalyst composition according to claim 38, wherein the phosphine ligand may form a bi-dentate ligand with respect to the Group VIII metal.

40. A catalyst composition according to claim 38, wherein the heteroatom comprises one or more selected from the group comprising O, S, N and P.

41. A catalyst composition according to claim 40, wherein the heteroatom comprises one or more selected from the group comprising N and P.

42. A catalyst composition according to claim 36, wherein only one of R¹, R², and R³ include a heteroatom-substituted hydrocarbon group.

43. A catalyst composition according to claim 36, wherein the phosphine ligand has the formula R¹, R²P{link}PR⁶R⁷, wherein R¹, R², R⁶, and R⁷ are the same or different, and {link} is a direct link between the phosphine groups, or {link} is a group comprising a single atom linking the phosphine groups.

44. A catalyst according to claim 36, wherein the phosphine ligand comprises a diphosphine ligand having a formula selected from R¹, R²PCH₂PR⁶R⁷ and R¹, R²PN(R)PR⁶R⁷ wherein R¹, R², R⁶, and R⁷ are the same or different.

45. A catalyst composition according to claim 43, wherein R¹ and R² are the same and/or R⁶ and R⁷ are the same.

46. A catalyst composition according to claim 43, wherein R¹, R², R⁶ and R⁷ are independently selected from hydrogen, hydrocarbon groups and heteroatom-substituted hydrocarbon groups.

47. A catalyst composition according to claim 36, wherein the phosphine ligand has a formula selected from R₂PCH₂PR₂ and R₂PN(R)PR₂.

48. A catalyst composition according to claim 47, wherein R is selected from hydrogen, hydrocarbon groups and heteroatom-substituted hydrocarbon groups.

49. A catalyst composition according to claim 36 wherein the catalyst comprises a phosphine ligand having the formula R¹ R²P{link}NR⁶R⁷, wherein R¹, R², R⁶, and R⁷ are the same or different, and {link} is a direct link between the phosphine group and the nitrogen donor group.

50. A catalyst composition according to claim 36 wherein the catalyst comprises a phosphine ligand comprising a P˜N ligand having a formula selected from R¹, R²P(CR₂)ₙNR⁶R⁷ wherein R¹, R², R⁶, and R⁷ are the same or different and wherein n is 1, 2, 3 or 4.

51. A catalyst composition according to claim 49, wherein R¹ and R² are the same and/or R⁶ and R⁷ are the same.

52. A catalyst composition according to claim 49, wherein R¹, R², R⁶ and R⁷ are independently selected from hydrogen, hydrocarbon groups and heteroatom-substituted hydrocarbon groups.

53. A catalyst composition according to claim 50, wherein R is selected from hydrogen, hydrocarbon groups and heteroatom-substituted hydrocarbon groups.
